(19) United States
(12) Patent Application Publication
(10) Pub. No.: US 2011/0009762 A1
(43) Pub. Date: Jan. 13, 2011

(54) PORTABLE PNEUMOTACHOGRAPH FOR MEASURING COMPONENTS OF AN EXPIRATION VOLUME AND METHOD THEREFOR

(75) Inventors: Ruediger Eichler, Zellingen (DE); Stefan Dietze, Lehnitz (DE); Werner Steinhaeusser, Kirchheim (DE); Gunther Becher, Bernau (DE)

Correspondence Address: PATENT CENTRAL LLC;Stephan A. Pendorf, 1401 Hollywood Boulevard, Hollywood, FL, 33020, US

(73) Assignee: FILT Lungen-und Thoraxdiagnostik GmbH, Berlin (DE)

(21) Appl. No.: 12/530,227

(22) PCT Filed: Mar. 7, 2008
(86) PCT No.: PCT/DE2008/000417

§ 371 Date: Jul. 20, 2010

(30) Foreign Application Priority Data
Mar. 8, 2007 (DE) 10 2007 012 210.3
Mar. 8, 2007 (DE) 10 2007 012 285.5
Mar. 8, 2007 (DE) 20 2007 003 817.8
Mar. 8, 2007 (DE) 20 2007 003 818.6

(51) Int. Cl. A61B 5/08

(52) U.S. Cl. 600/532

(57) ABSTRACT

A portable pneumotachograph for determining components of the expiration volume, comprising a processor (3), a PEEP valve (4) mounted on the pneumotachograph (1) on the exhaling side, a filter (5) mounted on the pneumotachograph (1) on the inhaling side for removing the portion of the component in the inspiration air to be determined, at least one sensor (7) mounted in or on the pneumotachograph tube (6), wherein in the sensor (7) disposed on the pneumotachograph tube (6), the pneumotachograph tube (6) having an opening (8) on the sensor side, and/or at least one means configured in the pneumotachograph tube (6) for sampling, and further an optical and/or acoustic control device (9) for the expiration flow. This enables a correlation of measurement data of one or more components of the exhaled flow detected with a lung function test, enabling the localization of the seat of the disease.

The invention relates to a portable pneumotachograph and a method for the measurement of components of the expiration flow, particularly of NO.

Nitrogen oxides and other gaseous compounds in the exhaled air are used to assess the physical condition of human beings, because they are indicators of metabolic processes in the body, disorders and diseases in humans. Inpatient diagnostic equipment for gas analysis of exhaled air is known and has been available on the market for a long time.

A portable gas analyzer with an NO-sensor is described in EP 1 439 781, in which the patient breathes at a prescribed flow rate and a prescribed pressure. A disadvantage of the device described there is that no spirometric data can be collected, which would allow a correlation of the measurements to the afflicted parts of the lungs.

Also devices for lung function analysis, spirometry, have been in the use for years.

Spirometry is a method for lung function testing. The lungs- and respiratory volume is measured and plotted in spirogramms. To obtain data on lung volumes a spirometer or pneumotachograph is needed.

The patient breathes through a mouthpiece into a breathing tube while the nose is closed with a nose clip. The spirometer measures electronically through a flow sensor the airflow speed at which the air is inhaled and exhaled, from which the amount of air breathed per time unit is calculated. The amount of air that is moved by these breaths is graphically displayed by the device. Thus a direct comparison of the measurements from different tests can be made.

Through measurement of the airflow velocity or expiration velocities and the lung volumes, the physician can diagnose diseases of the lungs and control the aetiopathology. The following values can be measured using spirometry:

Tidal Volume (TV): The tidal volume corresponds to the volume in- or exhaled during normal breathing.

Inspiratory Reserve Volume (IRV): This is the volume that can be inhaled by forcible inspiration after completion of a normal inspiration.

Expiratory Reserve Volume (ERV): This is the volume that can be exhaled by forcible expiration after completion of a normal expiration.

Inspiratory Capacity (IC): It is defined as the volume that can be maximally inhaled after a normal exhalation.

Vital Capacity (VC) is the volume that can be maximally exhaled after maximal inhalation.

Forced Expiratory Capacity in 1 Second (FEV1, Tiffeneau test) is the maximum volume that can be forcibly exhaled within one second after maximum inspiration.

These metrics help for example to distinguish between the two main groups of lung diseases:

Obstructive pulmonary diseases: These are caused by a constriction of the airways, e.g. asthma or COPD.

Restrictive lung diseases: Here the ability to expand the lung and/or ribcage is decreased. Examples include hardening of the lungs (pulmonary fibrosis), fluid accumulation in the oblique fissure of the lung (pleural effusion) or a high-standing diaphragm (diaphragm paresis).

In spirometry, the patient breathes in respectively out through a mouthpiece. The mouthpiece is connected to a spirometer and in most cases equipped with a bacterial filter. To obtain the different measured variables, the patient must accurately follow the instructions of the examiner on breathing in and out. Otherwise, incorrect values are measured, which in turn can lead to incorrect conclusions for the treatment. The investigation thus depends on a good cooperation of the patient.

Another device for the measurement of NO in the expiration volume is described in U.S. Pat. No. 6,010,459. Here the spirometry is conducted after measuring the values. The patient breathes a synthetic mixture of humidified air and NO. When exhaling, the patient must generate a defined pressure in the measurement device, ostensibly so that the soft palate closes during expiration, to block air from the nasal cavity in which the NO concentration can be up to 100 times compared to the exhaled air and which would distort the measurement from getting into the expiration volume. A disadvantage of this device is the lack of portability, since it needs synthetic gas to be inhaled for the measurement.

Further, with the ATS/ERS Guidelines published in 2005 (Exhaled breath condensate: methodological recommendations and unresolved questions. I. Horvath, J. Hunt and P. J. Barnes, On behalf of the ATS/ERS Task Force on Exhaled Breath Condensate, Eur Respir J 2005; 26: 523-548) there is available for the first time a comprehensive report on the methods of breath condensate diagnostics. Guidelines for NO measurement are published in: “ATS Workshop Proceedings: Exhaled Nitric Oxide and Nitric Oxide Oxidative Metabolism in Exhaled Breath Condensate: Executive Summary. Am J Respir Crit Care Med 2006 Apr. 1; 173 (7) :811-813” and “American Thoracic Society Documents: ATS Workshop Proceedings: Exhaled Nitric Oxide and Nitric Oxide Oxidative Metabolism in Exhaled Breath Condensate. Proc Am Thorac Soc Vol 3rd pp 131-145, 2006”.

It should be stated that the standardization of sample collection, sample storage and analysis is still to be improved. The standardization of sampling must be done in the future as well. Thus, especially in the analysis the procedures used and their validation need more attention.

The objective of the invention to create a pneumotachograph for measuring components of the expiration volume which is portable and handy, and that allows for a correlation of collected data of one or several components of the expiration flow with a lung function test, where the analysis can be carried out in compliance with standardizing guidelines and allows a localization of the disease.

The objective is accomplished by the invention with a portable pneumotachograph to determine components of the expiration volume, which features a processor, a PEEP valve on the expiration side of the pneumotachograph, a filter on the inspiration side of the pneumotachograph for the removal of the portion of the component to be measured in the inhaled air, at least one sensor mounted in or on the pneumotachograph tube, with an opening of the pneumotachograph tube on the sensor side and/or at least one means for sampling in the pneumotachograph tube and a visual and/or acoustic control of the expiration flow.

The core of the portable pneumotachograph to determine components in the expiration volume is a pneumotachograph, preferably with an exchangeable mouthpiece with an inserted bacterial filter. The patient breathes in and out through the pneumotachograph. On the inspiration side a filter is attached to the pneumotachograph to filter out the component to be measured from the surrounding air. Inside the pneumotachograph in the direction of the exhalation flow behind the lamellae or grids installed in the pneumotachograph tube to produce a flow resistance, a sensor and/or sampling device is located for example in mid-stream or in the pipe wall, for example in the form of a cannula which preferably extends into the flow channel of the pneumotachograph tube. This means that in the first embodiment the sensor or the sensors or their sensitive layers are located directly in the mainstream and activation and/or release of the sensor measurement occurs preferably at the desired point time. To guard against contamination by saliva or other substances, the sensor or the sampling orifice is oriented in the flow direction of the expiration flow and shaped such that no saliva or potentially forming condensation water can enter the sampling orifice. The exhalation takes place against an expiratory resistance, which is produced by the PEEP valve mounted on the expiration side of the pneumotachograph tube. The expiratory resistance is preferably from 5 to 20 cm H₂O, and causes an increase in the mean airway pressure and the functional residual capacity.

By means of the PEEP valve it is achieved that the ventilation of the lungs respectively alveoli even with an airway obstruction, secretions in the airways or different kinds of ventilation disorders is done more evenly. This is a prerequisite for reproducible repetition of respiratory maneuvers and a largely undisturbed emission of the components of the exhaled air during the measuring process.

To reduce the risk of measurement errors by the addition of nasal air the following measures can be provided:
Wearing a nose clip during inspiration
Expiration with a constant flow respectively a flow greater than zero, because a halt of the flow during the expiration brings air from the nose into the throat.

A constant flow is a flow with a maximum deviation of +/−10% of the mean.

In order to control the flow, the portable pneumotachograph has an optical or acoustic means of control, by which the patient can control and set his or her expiration.

In a preferred embodiment a valve or a flap, which closes the inlet opening of the pneumotachograph during expiration is located between the filter that is mounted on the inspiration side of the pneumotachograph, which is exchangeable, and the pneumotachograph tube. This is to ensure that the patient is breathing only against the expiratory resistance imposed by the PEEP valve. Preferably, the valve or the flap closes automatically. Such a valve can be a check valve, a stop valve or a simple flap which opens into the pneumotachograph tube. But there are no limits with regard to the specific design of this valve.

The sensor can be selected from the group “electrochemical sensor, chemiluminescence sensor, NO sensor, O₂ sensor, H₂O₂ sensor, CO₂ sensor, CO sensor” and/or a combination sensor from the aforementioned sensors. Depending on the component of the exhaled air to be analyzed (NO, O₂, . . . ) the corresponding sensor needs to be chosen. It can also be a set up with multiple sensors for simultaneous measurement of several components.

In the opening between the sensor and pneumotachograph tube a closed loop controlled and/or open loop controlled valve, in the following called sensory valve, or a closed loop controlled or open loop controlled pump, in the following called sensory pump, can be installed. To this end, the sensor can be set up outside the pneumotachograph tube and be connected to the pneumotachograph tube through a line and a sample. The sensory valve or the sensory pump may be in a position “open” or “closed” according to the requirements of the inspiration or expiration volume or flow or of a partial inspiration or partial expiration volume or flow that is passing through the pneumotachograph tube, whereas preferably the sensory valve is controlled by the processor. Hereby it is achieved that the sampling respectively measurement is performed only when defined volume flows or partial volume flows pass through the expiration pneumotachograph tube. The timing of when a measurement has to be made is calculated with the flow-volume correlation determined by the pneumotachograph, which is connected to a processor, to and by which also the sensory valve is connected and controlled. Since the sampling is done directly from the processor controlled pneumotachograph, any delay or discordance between flow measurement and sampling can be ruled out.

The optical control of the expiratory stream can be selected from the group “y-t graph, bar graph, LED display with one or more light emitting diodes”. The acoustic monitoring can be a beep or a sound which is changing in volume or frequency.

In a particular embodiment, the PEEP valve is a double valve, which limits the flow between a minimum flow and a maximum flow. In other words, the valve opens when reaching an initial expiration pressure which is in correlation to the minimum flow, such that the flow velocity in the pneumotachograph tube steeply increases from a value of “zero” to the flow velocity of the minimum flow. The patient can then breathe out with a defined flow. When exceeding a second expiration pressure that is higher that the first and which correlates with the maximum flow the valve closes again and the value for the flow velocity drops sharply to a value of “zero”. The patient is now prevented from exhaling. Expiration is only possible between the minimum flow and the maximum flow. Such a PEEP—double valve exhibits for example on the inlet side (the side of the pneumotachograph) a spring-loaded check valve and on the outlet side (ambient) a pressure valve, such that the spring-loaded check valve will only open when reaching or exceeding a first pressure value on the input side and the pressure valve on the outlet side closes when exceeding a higher back pressure generated on the input side that is greater than the first pressure (opening pressure). In other words, the PEEP valve opens its inlet aperture only when exceeding a first defined pressure resistance. From this moment on its output aperture is opened. When increasing the flow, the pressure onto the check valve increases at the same time and its spring will be compressed further and pushes toward the outlet opening. On the back side between the seal of the check valve and the spring e.g. a cone is attached which gradually closes the outlet opening when the spring is compressed. With this kind of PEEP valve the expiratory flow and expiratory resistance can be limited between an adjustable minimum and an adjustable maximum value.

The portable pneumotachograph may include one or more gas-impermeable collection container for collecting samples and/or respiratory volumes of several breaths. With these several breaths can be collected or samples can be stored until analysis.

The surfaces of the sample conducting tubing or air ducts of the pneumotachograph according to the invention may be modified and be such that in these membranes, liquid films applied or inlay of porous layers or membranes are incorporated, so that certain components of the sample gas are retained or chemically bound in the layers. This list is exemplary and not limiting. For example, the water vapor content of the sample can be reduced by hygroscopic substances or water-binding layers or substances or chemicals from the expiration flow or inspiration flow that would interfere with the analysis can be bound physically or chemically, to ensure an accurate analysis.

The deposition of water vapor on the sensor or sensors and/or in the tubing/ducting to the sensors can also be prevented by temperature control to a temperature of 35 to 40° C. corresponding to that of the exhaled air.

Free radicals on the other hand can be deactivated by chemical reaction in or on the modified surfaces such that the sample is stabilized. In addition elements of the expiration flow which are available in a form that cannot be analyzed or in a superimposed form through cross-reactions can be converted into a selectively detectable form. The modification of the surfaces of the tubing or air ducts that the sample flows through can also be done by a biological immobilizate, for example an immobilized enzyme that specifically converts the organic compounds or specifically binds components.

The portable pneumotachograph can be provided with a purging device, which allows that the sensor and/or sampling is purged and thus cleaned of the exhaled air with a gas selected from the group “component free air, synthetic air, for the purpose of calibration produced gases, or a combination of these gases”, in order to increase the accuracy of the measurements. To this end, a pump can be set up, that, for example, with a filter which is preferably connected with a separate tube/duct to the inspiration side of the pneumotachograph, pumps ambient air through the filter into the sampling collection or sensor area. The purging air can be disposed of through the PEEP valve to the environment or through a separate purge gas line. However, it may also exhibit one or more ports for connecting up to a pressurized or pressure free purge gas container.

In a further embodiment, a calibration of the sensor with a gas selected from the group “component free air, synthetic, for the purpose of calibration produced gases, or a combination of these gases” was established. The calibration values of these gases are individually adjustable with the device.

Furthermore, the invention teaches a method for the determination of components of the expiration flow consisting of the following steps:
a) Filtering the component to be determined or the components to be determined from the inhaled air.
b) Measuring the inspiration volume.
c) Generating a positive pressure in the lung against a pressure resistance which is generated by a closed pressure valve.
d) Open the pressure valve by overcoming the pressure resistance.
e) Generating a defined expiratory flow at a pressure level higher than the predetermined pressure resistance.
f) Discounting the dead space air of the mouth and throat.
g) Sampling of expiratory air and/or sensory measurement of the component or the components to be determined in the expiratory air after discounting the dead space air.

In practice the procedure, for example for a NO analysis, is as follows: The patient is asked to exhale deeply. Immediately thereafter, the patient must breathe in deeply with a mouthpiece filter through the pneumotachograph according to the invention. This inspiration is done for example through an activated carbon filter (breathing filter) through the pneumotachograph into the lungs.

This breath is recorded and the expected expiration volume is calculated using the inspiration volume. Then the patient must slowly exhale deeply without removing the mouthpiece.

This breath should occur, against an expiratory resistance preferably in the range 5 to 20 cm H₂O, as predetermined by the analyzer and induced by the PEEP valve. With a control system (optical or acoustic control) the patient is shown the expiratory flow, preferably as a y-t graph on a screen. Alternatively a bar graph or different colored LEDs may be used. The acoustic control can be a beep or sound changing in the volume or frequency.

The expiratory flow should be preferably 50 ml/sec. This can also be varied. The flow of 50 ml/sec should be maintained within a range of +/−10% for 4 sec for children under 12 years or 6 sec in children over 12 years and adults. At a flow of 50 ml/sec this corresponds to a total of about 300 ml air.

The adaptation to the flow rates and pressures required for the analysis of one or more components of the exhaled air, for example because of required boundary conditions by law or regulations is carried out by the selection and use of a PEEP valve or a controllable PEEP valve that delivers the required flow and pressure by adjusting to the required the parameters.

During this plateau the measured NO value should remain in a range of +/−10%.

The measurement is to be repeated. The measurement is subject to the ATS/ERS guidelines if at least two breathing maneuvers match the criteria.

Another possible process step may be that a part of and/or the entire expiration volume is collected in a gas-impermeable collection container, preferably a gas sample bag.

The sampling and/or the sensor can further be purged with one or more gases selected from the group “component free air, synthetic, for the purpose of calibration produced gases or a combination of these gases” after each measurement.

A calibration of the sensor can be done with one or more gases selected from the group “component free air, synthetic, for the purpose of calibration produced gases or a combination of these gases” after one or a number of measurements.

The component free air can be generated by pumping ambient air through the air filter mounted on the inspiration side of the pneumotachograph. This purified air is then routed over the sensor.

The constant flow of expiratory air is preferably chosen in the range of 10-500 ml/s, particularly from 45 to 55 ml/s.

The expiratory flow needs to be kept constant for a period of 1 to 30 s, preferably from 2 to 10 s, particularly preferred from 4 to 6 s.

Based on the value of the inspiration volume and a corresponding mathematical correlation to the expected expiration volume an attribution of the measurement values to the sequentially exhaled partial volume flows of an expiration flow is possible, while at the same time these partial volume flows may be attributed to certain regions and zones of the respiratory tract. Through this operation, diseases and disorders of the respiratory tract can be localized.

Another process step stipulates that a measurement of components of the expiration volume is only performed when a defined partial expiration volume passes the sensor. With the spirometric data acquisition the processor can calculate the point in time at which a partial volume flow x, originating from an area Y of the lung, in which for example the seat of a disease is suspected, passes the sensor. A measurement of the components only of this partial volume flow can then be performed.

In accordance with standards, for example the ATS/ERS guidelines, only those measurements are considered analyzable and representative, which were obtained under defined measurement conditions. To this end the method according to the invention ensures that a measurement of constituents of the expiration flow is only performed when the specified parameter or parameters “overcoming the expiratory resistance” and/or “constant expiratory flow” and/or “duration of the expiratory flow” are given. For the case these parameters are not met, or not met for a sufficient length of time, no measurement is taken, i.e. the sensor receives no signal from the processor to initiate a measurement. Insofar the sensor is not activated or a necessary condition after the initiation of the sensory measurement was not met, a measurement that was performed under non-standard conditions can be identified and displayed as such.

Preferably the values of the parameters “overcoming the expiratory resistance” and/or “constant expiratory flow” and/or “duration of the expiratory flow” are individually adjustable and/or selectable through a memory device depending on the patient group and/or the condition of the patient's expiratory tract.

In a particular embodiment, the PEEP valve is removable. With that the portable pneumotachograph can also be used for the measurement of spirometric data without the need to provide an additional spirometer. For the time of acquisition of the spirometric measurements, the measurement sensor is preferably disabled or turned off.

In the following embodiments of the invention will be explained in more detail with reference to the illustrations below. They show:

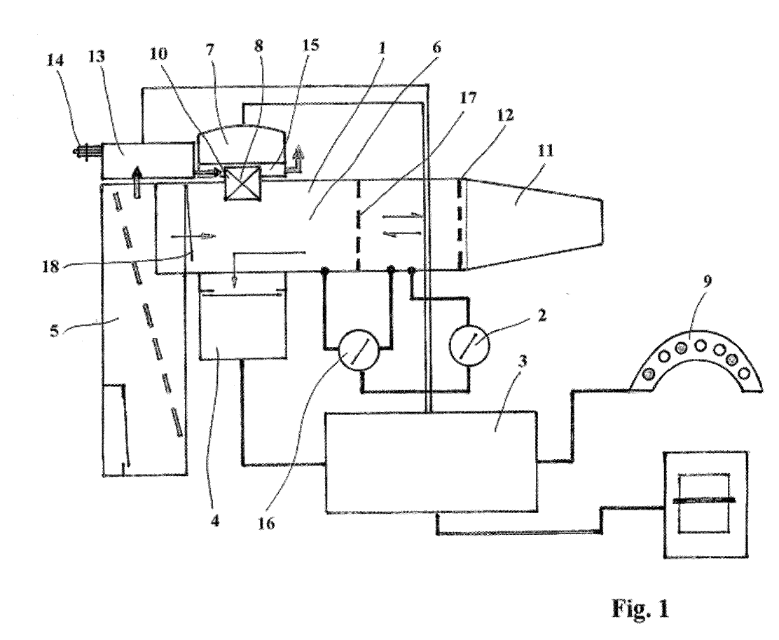

FIG. 1: schematic representation of the portable pneumotachograph,

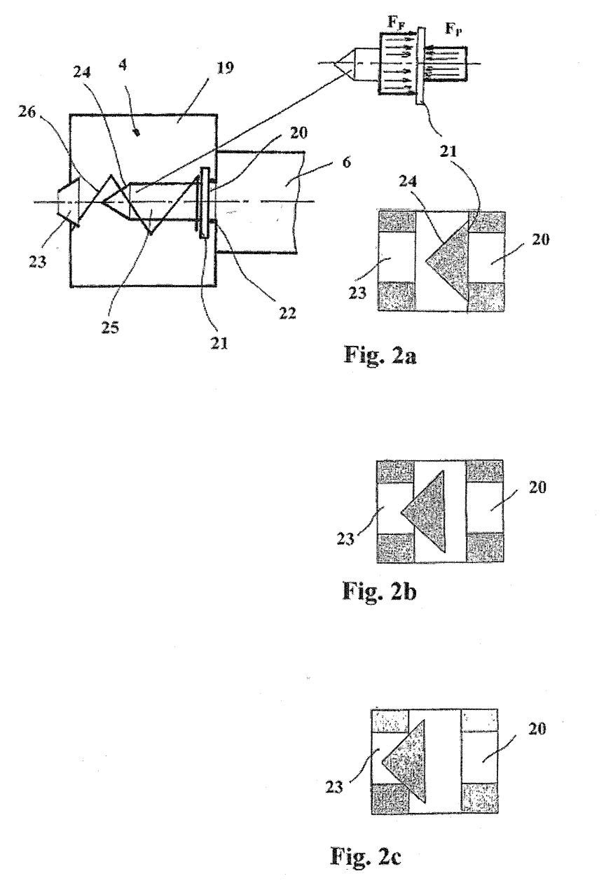

FIG. 2: a PEEP-valve and

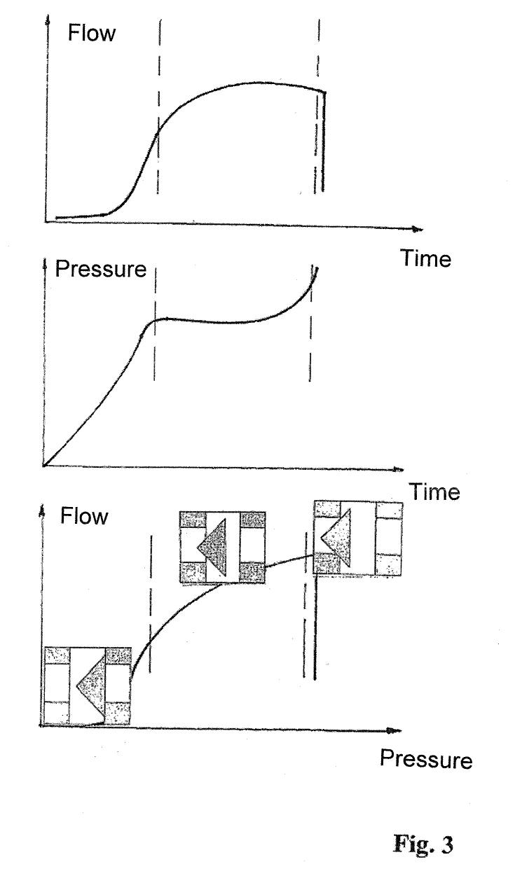

FIG. 3: characteristics of the PEEP-valve

FIG. 1 shows a portable pneumotachograph for measuring components of an expiration volume with a pneumotachograph 1 with a means for pressure measurement 2, and a processor 3, a PEEP valve 4 connected to the expiration side of the pneumotachograph 1, a filter 5 connected to the inspiration side of the pneumotachograph 1 for the removal of the fraction of the component to be determined in the inhaled air, a sensor 7 connected to the pneumotachograph tube 6, wherein the pneumotachograph tube 6 features opening 8 on the sensor side, in which a flow controller 10 is located, and an optical control 9 of the expiration flow. The mouthpiece 11 is equipped with a bacterial filter 12. To be able to purge the sensor 7, the pump 13 (with an additional connection 14 for connecting a purge gas container) pumps the purge gas, in this case ambient air that is purified from the component to be measured, through the filter 5 into the sensor chamber 15. According to the standard the pneumotachograph features an electric pressure gauge 16 which measures the pressure difference in front of and behind the lamellae 17.

Between the filter 5 installed on the inspiration side of the pneumotachograph 1 and the pneumotachograph tube 6, a flap 18 is installed, which closes the inlet opening of the pneumotachograph on expiration.

The PEEP valve 4 shown in FIG. 2a-c comprises of a housing 19 with an inlet opening 20 of the pneumotachograph tube 6 and an outlet opening 23 as well as the on the inside of the housing 19 in front of the inlet opening 20 located a valve plate 21 with the valve seat 22 at the inlet opening 20 constituting a check valve. The valve plate 21 is pushed against the valve seat 22 by the compression spring 26, which is supported by the housing wall opposite the inlet wall, and seals off the inlet opening 20. If the force F_{P} produced by the PEEP in the pneumotachograph tube 6 reaches a value greater than the spring force F_{F} the valve plate 21 opens the inlet opening 20 and the flow from the pneumotachograph tube 6 may pass through against the resistance of the PEEP valve 4 and exit through the outlet opening 23.

If the flow increases and thus the PEEP and therefore the force F_{P} then the valve plate 21 will move further away from the valve seat 22 until the valve head 24 on the back of the valve plate 21, which together with the outlet opening 23 forms a pressure valve, closes. Thus the flow drops to the value 0, i.e. no or only a small quantity of air under great pressure can be blown into pneumotachograph tube 6 by the patient.

With the arrangement of the valve head 24 through a shaft 25 on the valve plate 21 and the possibility to adjust the shaft length, there is a functional relationship between PEEP and closing of the outlet opening 23, such that the boundaries can be adjusted for optimum flow. This purely mechanical adjustment through adjusting the length of the shaft is of course only one technical possibility.

Likewise other types of valves such as flap valves or diaphragm valves can be utilized without the need to be explained here in detail.

This mechanism exhibits—as can be seen from the curves shown in FIG. 3—the advantage that a flow is only possible within a pre-determined adjustable range. The lower limit is set by the opening resistance between the valve plate 21 and valve seat 22 and the upper limit with the force couple F_{F}<<F_{P}, is also adjustable. If this limited flow is aligned with the sensor system according to the invention then measurements can be realized under optimum flow conditions.

### REFERENCE NUMBER LIST

1 pneumotachograph

2 means for measuring pressure

3 processor

4 PEEP valve

5 filter to remove the fraction of component to be determined in the inhaled air

6 pneumotachograph tube

7 sensor

8 opening

9 optical control

10 flow controller

11 mouthpiece

12 bacterial filter

13 pump

14 connection

15 sensor chamber

16 electrical pressure gauge

17 lamellae

18 flap

19 PEEP valve housing

20 inlet opening

21 valve plate

22 valve seat

23 outlet opening

24 valve head, preferably conical shape

25 shaft of the conical valve head

26 pressure spring

1. A portable pneumotachograph for measuring components of an expiration volume consisting of a pneumotachograph (1) having an inspiration side and an expiration side, a processor (3), a PEEP valve (4) that is connected to the expiration side of the pneumotachograph (1), a filter (5) to remove the fraction of component to be determined in the inhaled air that is connected to the inspiration side of the pneumotachograph (1), a pneumotachograph tube (6), at least one sensor (7) mounted in or on the pneumotachograph tube (6), wherein for the case of the sensor (7) is mounted on the pneumotachograph tube (6) the pneumotachograph tube (6) features an opening on the sensor (8) side and/or at least one means to collect a sample that is located in the pneumotachograph tube (6) and furthermore a visual and/or acoustic control (9) of the expiration flow.

2. The portable pneumotachograph according to claim 1, wherein between the filter (5) that is connected to the inspiration side of the pneumotachograph (1) and the pneumotachograph tube (6) a valve (18) is configured, which closes the inlet opening pneumotachograph (1) during expiration.

3. The portable pneumotachograph according to claim 1, wherein the sensor (7) is selected from the group electrochemical sensor, chemiluminescence sensor, NO sensor, O₂ sensor, H₂O₂ sensor, CO₂ sensor, CO sensor, sensor for biomarkers or a combination of sensors from the aforementioned sensors.

4. The portable pneumotachograph according to claim 1, wherein, in the opening (8) between the sensor (7) and the pneumotachograph tube (6), a closed loop controllable and/or open loop controllable valve (15) is installed.

5. The portable pneumotachograph according to claim 4, wherein the valve (15) is “open” or “closed” according to the requirements of the inspiration or expiration volume or of a partial expiration volume that is passing through the pneumotachograph tube (6), whereas optionally the valve (15) is controlled by the processor (3).

6. The portable pneumotachograph according to claim 1, wherein the visual control (9) of the expiratory flow is selected from the group y-t graph, bar graph, and LED display with one or several light-emitting diodes.

7. The portable pneumotachograph according to claim 1, wherein through the PEEP valve (4) an expiratory resistance, preferably from 5 to 20 cm H₂O, is adjustable and producible.

8. The portable pneumotachograph according to claim 1, that wherein the PEEP valve (4) exhibits on the inlet side of the pneumotachograph a spring-loaded check valve and on the outlet side (ambient) a pressure valve, such that the spring-loaded check valve will open when exceeding a first pressure value on the input side and the pressure valve on the outlet side closes when exceeding a second pressure value on the input side that is greater than the first pressure value.

9. The portable pneumotachograph according to claim 8, wherein the check valve consists of a valve plate (21) that pushes with a force (F_{F}) against the flow direction in the pneumotachograph tube (6) that seals the valve seat (22) and is located in front of an inlet opening (20) with the valve seat (22) in the PEEP valve housing (19) and the valve plate (21) with a valve head (24) for the outlet opening (23) is coupled such that a definable value of the opening between valve seat (22) and valve plate (21) causes the complete or nearly complete closure of the outlet opening (23) with the valve head (24).

10. The portable pneumotachograph according to claim 8, wherein the valve head (24) is shaped conical, or parabolic, or convex or in the form of a sequential combination of the aforementioned shapes.

11. The portable pneumotachograph according to claim 8, wherein the valve head (24) is connected with the back of the valve plate (21) by a shaft (25).

12. The portable pneumotachograph according to claim 8, wherein the transfer of the dimension of the opening of the valve plate (21) on the movement of the valve head (24) is adjustable up to the closure of the outlet opening (23).

13. The portable pneumotachograph according to claim 8, wherein the valve plate (21) on the valve seat (22) has one of the following configurations
hinged flap sealing, so that the opening takes place with a changing angle between the valve plate plane and the valve seat plane,
parallel closing sealing, so that the opening takes place while maintaining the parallelism between the valve plate plane and the valve seat plane,
membrane sealing, wherein the membrane deforms under PEEP and thereby opens the outlet opening.

14. The portable pneumotachograph according to claim 8, wherein the PEEP valve housing (19) features a further air outlet opening to ensure a residual flow under the condition of a completely closed outlet opening (23).

15. The portable pneumotachograph according to claim 1, wherein one or more collection containers is provided for the collection of samples and/or respiratory volumes of several breaths.

16. The portable pneumotachograph according to claim 1, further comprising means for purging the sensor (7) and/or the sampling with a gas selected from the group component free air, synthetic, gases produced for the purpose of calibration, or a combination of these gases.

17. The portable pneumotachograph according to claim 1, wherein the sensor (7) can be calibrated with a gas selected from the group component free air, synthetic, gases produced for the purpose of calibration, or a combination of these gases.

18. The portable pneumotachograph according to claim 1, wherein the PEEP valve (4) is removable.

19. The portable pneumotachograph according to claim 1, wherein the surfaces of the sample conducting tubing or air ducts of the pneumotachograph are modified for the conversion and/or binding of components of the breathable air such that the surface itself is physically and/or chemically active or physically and/or chemically active membranes, liquid films, porous layers, or biological immobilizates are applied and/or incorporated on and/or in the surfaces individually or in combination.

20. The portable pneumotachograph according to claim 1, wherein the sensor (7) or the sensitive layer of the sensor (7) is located in the pneumotachograph tube (6), in the main-stream.

21. The portable pneumotachograph according to claim 20, wherein the sensor (7) features a control that allows to switch off and/or release the sensor (7) for a measurement at the desired point in time.

22. A method for measuring components of an expiration volume by means of a portable pneumotachograph according to claim 1 with the following process steps:
a) filtering the component to be determined or the components to be determined from the inhaled ail;
b) measuring the inspiration volume;
c) generating a positive pressure in the lung against a pressure resistance which is generated by a closed pressure valve;
d) open the pressure valve by overcoming the pressure resistance;
e) generating a defined expiratory flow at a pressure level higher than the predetermined pressure resistance;
f) discounting the dead space air of the mouth and throat; and
g) sampling of expiratory air and/or sensory measurement of the component or the components to be determined in the expiratory air after discounting the dead space air.

23. The method for measuring components of an expiration volume according to claim 22, wherein the component or the components to be determined are selected from the group NO, nitrogen, oxygen, free radicals, CO, H₂O₂ and other biomarkers.

24. The method for measuring components of an expiration volume according to claim 22, wherein components are filtered from the inspiration flow that are chosen from the group solid particles, dust particles, aerosols, and water vapor.

25. The method or measuring components of an expiration volume according to claim 22, wherein a partial and/or the entire expiration volume is collected in a gas-impermeable collection container.

26. The method for measuring components of an expiration volume according to claim 22, wherein the sampling and/or the sensor (7) are purged with one or more gases selected from the group component free air, synthetic, gases produced for the purpose of calibration, or a combination of these gases after each measurement unit.

27. The method for measuring components of an expiration volume according to claim 22, wherein the sensor (7) is calibrated after each or a number of measuring units with one or more gases selected from the group component free air, synthetic, gases produced for the purpose of calibration, or a combination of these gases.

28. The method for measuring components of an expiration volume according to claim 26, wherein component free air is generated by taking in ambient air through the air filter (5) mounted on the inspiration side of the pneumotachograph.

29. The method for measuring components of an expiration volume according to claim 22, wherein the constant flow of expiratory air is 10-500 ml/s, in particular 45 to 55 ml/s.

30. The method for measuring components of an expiration volume according to claim 22, wherein the expiratory resistance in pneumotachograph is in the range from 5 to 20 cm H₂O.

31. The method for measuring components of an expiration volume according to claim 22, wherein the expiratory flow is kept constant for a period of 1 to 30 s.

32. The method for measuring components of an expiration volume according to claim 22, wherein based on the inspiration volume an attribution of the sequentially exhaled partial volume flows of an expiration flow is done, and wherein these partial volume flows are attributed to certain regions and zones of the respiratory tract.

33. The method for measuring components of an expiration volume according to claim 22, wherein a measurement of components of the expiration flow only takes place when a defined partial expiratory volume is passing the sensor (7).

34. The method for measuring components of an expiration volume according to claim 22, wherein a measurement of components of the expiration flow is only performed when the specified parameter or parameters “overcoming the expiratory resistance” and/or “constant expiratory flow” and/or “duration of the expiratory flow” of the expiration volume are given.

35. The method for measuring components of an expiration volume according to claim 22, wherein the values of the parameters “overcoming the expiratory resistance” and/or “constant expiratory flow” and/or “duration of the expiratory flow” are individually adjustable and/or selectable from a memory device depending on the patient group and/or the condition of the patient's expiratory tract.

36. The method or measuring components of an expiration volume according to claim 22, wherein the deposition of water vapor on the sensor or sensors and/or in the tubing/ducting to the sensor or sensors is prevented by controlling the temperature of the exhaled air preferably by maintaining a temperature of 35 to 40° C.
